(19) United States
(12) Patent Application Publication
(10) Pub. No.: US 2006/0149242 A1
(43) Pub. Date: Jul. 6, 2006

(54) Spinal stabilization systems supplemented with diagnostically opaque materials

(76) Inventors: Gary Kraus, Houston, TX (US); Jamal Taha, Cincinnati, OH (US)

Correspondence Address: DINSMORE & SHOHL LLP;Suite 1300, One Dayton Centre, One South Main Street, Dayton, OH, 45402-2023, US

(21) Appl. No.: 11/303,750

(22) Filed: Dec. 16, 2005

Related U.S. Application Data
(60) Provisional application No. 60/637,432, filed on Dec. 17, 2004.

(51) Int. Cl. A61F 2/30

(52) U.S. Cl. 606/61

(57) ABSTRACT

According to the present invention, spinal stabilization systems are supplemented with a diagnostically opaque material to enable the diagnostic recognition of the components of these systems. These spinal stabilization systems may comprise one or more flexible spacers, and one or more elastic cords, either or both of which may comprise a diagnostically opaque material. Additional spinal stabilization systems are contemplated.

### CROSS-REFERENCE TO RELATED APPLICATIONS

This application claims the benefit of U.S. Provisional Application Ser. No. 60/637,432, filed Dec. 17, 2004.

### BACKGROUND OF THE INVENTION

The present invention relates to spinal stabilization systems and, more specifically to improvements to spinal stabilization systems that include components that are diagnostically transparent.

### BRIEF SUMMARY OF THE INVENTION

According to the present invention, spinal stabilization systems are supplemented with a diagnostically opaque material to enable the diagnostic recognition of the components of these systems. These spinal stabilization systems typically comprise pedicle screws, at least one spacer, and at least one cord.

In accordance with the present invention, pedicle screws are implanted into vertebral pedicles of a patient's spine. Positioned in a space existing between adjacent implanted pedicle screws is a spacer. The spacer may be constructed of a tensile material that provides a degree of flexibility sufficient to enable the spacer to compress with corresponding extension of the spine and a degree of tensile strength sufficient to provide supportive resistance to the spinal extension. The spacer typically comprises a longitudinal axial channel through which a cord passes.

This cord generally is configured to, and provided in a length sufficient to, pass through this longitudinal axial channel. The cord may be constructed of a tensile material the same as or different from the tensile material of the spacers. The cord's tensile material provides a degree of longitudinal elasticity that enables the cord to lengthen with the flexion of the spine and shorten with extension of the spine, providing supportive resistance to the spinal flexion.

With the spinal stabilization system being internally implanted into the patient, there is a need for diagnostic recognition of the spacers and cord to ensure their structural integrity without the need for a surgical procedure. Therefore, the spacers and/or the cord comprise a diagnostically opaque material that enables their diagnostic recognition through advanced diagnostic imaging procedures. This allows for the periodic non-invasive monitoring of the structural integrity of the spacers and the cord.

In accordance with another embodiment of the present invention, a spinal stabilization system comprising pedicle hardware and supplemental spinal stabilization hardware is provided. The pedicle hardware is configured such that it can be mechanically coupled to the vertebral pedicles of a patient's spine. The supplemental spinal stabilization hardware is constructed of a tensile material and is configured to be mechanically responsive to corresponding extension and flexion of the spine. The supplemental spinal stabilization hardware comprises a material that is more diagnostically opaque than the tensile material.

Accordingly, it is an object of the present invention to supplement spinal stabilization systems with a diagnostically opaque material to enable the diagnostic recognition of the components of the systems. Other objects of the present invention will be apparent in light of the description of the invention embodied herein.

### BRIEF DESCRIPTION OF THE SEVERAL VIEWS OF THE DRAWINGS

The following detailed description of specific embodiments of the present invention can be best understood when read in conjunction with the following drawings, where like structure is indicated with like reference numerals and in which:

FIG. 1 is a schematic illustration of a spinal stabilization system bound to a patient's spine viewed posteriorly; and

FIG. 2 is a schematic illustration of a spinal stabilization system bound to a patient's spine viewed laterally.

### DETAILED DESCRIPTION

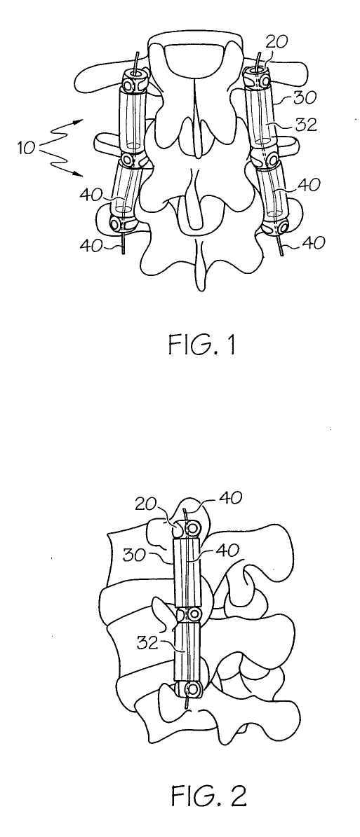

A spinal stabilization system 10 is illustrated schematically in FIGS. 1 and 2. In each Fig., the spinal stabilization system comprises pedicle screws 20, at least one spacer 30, and at least one cord 40. Referring to FIG. 1, pedicle screws 20 are implanted into vertebral pedicles of a patient's spine. These pedicle screws 20 are configured for such use and are typically constructed of a diagnostically opaque material. Positioned between adjacent pedicle screws 20 that have been implanted into the patient's spine is a spacer 30 that is configured to fit between such space so as to define at least a portion of the spacing separating adjacent pedicle screws 20.

The spacers 30 may be constructed of a tensile material. This tensile material provides a degree of flexibility sufficient to enable the spacer 30 to compress with corresponding extension of the spine and a degree of tensile strength sufficient to provide supportive resistance to the spinal extension. These spacers 30 typically comprise a longitudinal axial channel 32 through which a cord 40 passes.

This cord 40 generally is configured to, and provided in a length sufficient to, pass through the longitudinal axial channel 32 of the spacers 30. The cord 40 may be constructed of a tensile material that provides a degree of longitudinal elasticity that enables the cord to lengthen with the flexion of the spine and shorten with extension of the spine.

Thereby, the spinal stabilization system 10 is bound to the patient's spine with a degree of flexibility that enables the spine to assume positions of rest, flexion, and extension and with a degree of tensile strength that preserves the spine's natural anatomy and stabilizes the spine's degenerated segments in need of support. For the purposes of describing and defining the present invention, it is noted that spinal flexion refers to the general motion of the spine as the body bends forward while spinal extension refers to the general motion of the spine as the body returns to an upright orientation.

With the spinal stabilization system 10 being internally implanted into the patient and bound to the patient's spine, there is a need for diagnostic recognition of the components of the system 10 to ensure their structural integrity without the need for a surgical procedure. Therefore, the spacers 30 or the cord 40, or both, comprise a material that is more diagnostically opaque relative to the materials forming the spacers 30 and cord 40. This diagnostically opaque material is provided in sufficient quantity to enable diagnostic recognition of the spacers 30 or the cord 40, or both, thereby permitting the spacers 30 or the cord 40, or both, to be viewed through advanced diagnostic imaging procedures. This allows for the periodic non-invasive monitoring of the structural integrity of the spacers 30 while they are positioned between the vertebrae of the patient's spine and of the cord 40 while it passes through the longitudinal axial channel 32 of the spacers 30.

There are numerous ways to configure the spacers 30 or the cord 40 such that they comprise a diagnostically opaque material. For example, a diagnostically opaque material may be dispersed within, attached to, assembled as discrete parts of, embedded within, woven into, or coating the external areas of the tensile materials of the spacers 30 or the cord 40. None of these examples are intended as a limitation in providing diagnostically opaque material to the spacers 30 or the cord 40 for the diagnostically opaque material may be applied in any other manner to the tensile materials of the spacers 30 or the cord 40, or both, that allows for the diagnostic viewing and periodic monitoring of the structural state of the tensile materials.

Referring more specifically to the diagnostically opaque material of the present invention, it is noted that the material may be selected from a variety of diagnostically opaque materials, including materials that are opaque to X-ray diagnostics, CAT scanning, magnetic resonance imaging, or other radiological imaging techniques. Suitable materials include, but are not limited to surgical grade stainless steel, titanium, other metals and metal alloys, synthetic materials, carbon, graphite, combinations thereof, or any other suitable surgical material. It is contemplated that significant improvements in imaging operations can be achieved by ensuring that the diagnostically opaque material is about 50% more opaque than the tensile material, about twice as opaque as the tensile material, or at least one order of magnitude more opaque than the tensile material, depending upon the particular demands of the imaging technology at issue.

Referring specifically to the tensile material of the present invention, for example, and not by way of limitation, the spacers 30 typically may be constructed of a Sulene™ class substance, such as polycarbonate urethane. The cord 40, likewise, may be constructed of a Sulene™ class substance, such as polyethylene terepthalate. Thus, the tensile material of the cord 40 may be of a substance the same as or different from the tensile material of the spacers 30.

Although the present invention has been described in the context of a spinal stabilization system incorporating pedicle screws, tensile spacers, and tensile cords, it is contemplated that diagnostically opaque materials may be incorporated in a variety of spinal stabilization systems where one or more of the components of the system would not otherwise be diagnostically opaque.

It is noted that terms like “preferably,” “commonly,” and “typically” are not utilized herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention.

For the purposes of describing and defining the present invention it is noted that the term “substantially” is utilized herein to represent the inherent degree of uncertainty that may be attributed to any quantitative comparison, value, measurement, or other representation. The term “substantially” is also utilized herein to represent the degree by which a quantitative representation may vary from a stated reference without resulting in a change in the basic function of the subject matter at issue.

Having described the invention in detail and by reference to specific embodiments thereof, it will be apparent that modifications and variations are possible without departing from the scope of the invention defined in the appended claims. More specifically, although some aspects of the present invention are identified herein as preferred or particularly advantageous, it is contemplated that the present invention is not necessarily limited to these preferred aspects of the invention.

What is claimed is:

1. A spinal stabilization system comprising pedicle screws, at least one spacer, and at least one cord, wherein:
said pedicle screws are configured to be implanted into vertebral pedicles of a patient's spine;
said spacer is configured to be positioned between said pedicle screws implanted into said vertebral pedicles of said patient's spine so as to define at least a portion of a spacing between said pedicle screws;
said spacer comprises a longitudinal axial channel and is constructed of a tensile material that provides a degree of flexibility sufficient to enable said spacer to compress with corresponding extension of said spine and a degree of tensile strength sufficient to provide supportive resistance to said spinal extension;
said cord is configured to pass through said longitudinal axial channel of said spacer;
said cord is constructed of a tensile material that provides a degree of longitudinal elasticity that enables said cord to lengthen with said flexion of said spine and a degree of tensile strength sufficient to provide supportive resistance to said spinal flexion;
either said spacer or said cord or both comprise a material that is diagnostically opaque relative to said tensile material; and
said diagnostically opaque material is provided in sufficient quantity to enable diagnostic recognition of said spacer or said cord.

2. The spinal stabilization system of claim 1, wherein said spacer comprises said diagnostically opaque material provided in sufficient quantity to enable said diagnostic recognition of said spacer.

3. The spinal stabilization system of claim 1, wherein said cord comprises said diagnostically opaque material provided in sufficient quantity to enable said diagnostic recognition of said cord.

4. The spinal stabilization system of claim 1, wherein said spacer and said cord comprise said diagnostically opaque material provided in sufficient quantity to enable said diagnostic recognition of said spacer and said cord.

5. The spinal stabilization system of claim 1, wherein said spacer or said cord or both comprise said diagnostically opaque material dispersed within said tensile material.

6. The spinal stabilization system of claim 1, wherein said spacer or said cord or both comprise said diagnostically opaque material attached to said tensile material.

7. The spinal stabilization system of claim 1, wherein said spacer or said cord or both comprise said diagnostically opaque material assembled as a discrete part of said tensile material.

8. The spinal stabilization system of claim 1, wherein said spacer or said cord or both comprise said diagnostically opaque material embedded within said tensile material.

9. The spinal stabilization system of claim 1, wherein said spacer or said cord or both comprise said diagnostically opaque material woven into said tensile material.

10. The spinal stabilization system of claim 1, wherein said spacer or said cord or both comprise said diagnostically opaque material coating the external areas of said tensile material.

11. The spinal stabilization system of claim 1, wherein said diagnostically opaque material is more diagnostically opaque that said tensile material by at least one order of magnitude.

12. The spinal stabilization system of claim 1, wherein said diagnostically opaque material is at least twice as diagnostically opaque as said tensile material.

13. The spinal stabilization system of claim 1, wherein said diagnostically opaque material is at least 50% as diagnostically opaque as said tensile material.

14. A spinal stabilization system comprising pedicle screws, at least one spacer, and at least one cord, wherein:
said pedicle screws and are configured to be implanted into vertebral pedicles of a patient's spine;
said spacer is configured to be positioned between said pedicle screws implanted into said vertebral pedicles of said patient's spine so as to define at least a portion of a spacing between said pedicle screws;
said spacer comprises a longitudinal axial channel and is constructed of a tensile material that provides a degree of flexibility sufficient to enable said spacer to compress with corresponding extension of said spine and a degree of tensile strength sufficient to provide supportive resistance to said spinal extension;
said cord is configured to, and provided in a length sufficient to, pass through said longitudinal axial channel of said spacer;
said cord is constructed of a tensile material that provides a degree of longitudinal elasticity that enables said cord to lengthen with said flexion of said spine and a degree of tensile strength sufficient to provide supportive resistance to said spinal flexion;
said spacer or said cord or both comprise a material that is more diagnostically opaque relative to said tensile material;
said diagnostically opaque material is provided in sufficient quantity to enable diagnostic recognition of said spacer or said cord or both; and
said spacer or said cord or both comprise said diagnostically opaque material dispersed within, attached to, assembled as a discrete part of, embedded within, woven into, or coating external areas of said tensile material.

15. A spinal stabilization system comprising pedicle hardware and supplemental spinal stabilization hardware, wherein:
said pedicle hardware is configured to be mechanically coupled to vertebral pedicles of a patient's spine;
said supplemental spinal stabilization hardware is constructed of a tensile material and is configured to be mechanically responsive to corresponding extension and flexion of said spine;
said supplemental spinal stabilization hardware comprises a material that is diagnostically opaque relative to said tensile material; and
said diagnostically opaque material is provided in sufficient quantity to enable diagnostic recognition of said spacer or said cord.
